(19) United States
(12) Patent Application Publication
(10) Pub. No.: US 2022/0218593 A1
(43) Pub. Date: Jul. 14, 2022

(54) SKIN WHITENING, ANTI-BACTERIAL OR ANTI-ATOPIC COMPOSITION, CONTAINING SYZYGIUM FORMOSUM EXTRACT AS ACTIVE INGREDIENT

(71) Applicant: CARBOEXPERT INC., Daejeon (KR)

(72) Inventors: Chang Kyu LEE, Daejeon (KR); Min Jee YOO, Sejong (KR); Hyun Ah YU, Daejeon (KR); Jin Ju BAE, Daejeon (KR); Nan Young LEE, Daejeon (KR)

(21) Appl. No.: 17/285,066

(22) PCT Filed: Oct. 10, 2019
(86) PCT No.: PCT/KR2019/013306

§ 371 Date: Jan. 3, 2022

(30) Foreign Application Priority Data
Oct. 11, 2018 (KR) 10-2018-0121224

(51) Int. Cl. A61K 8/9789; A61K 36/61; A61P 17/00; A61Q 19/02

(52) U.S. Cl. CPC A61K 8/9789; A61Q 19/02; A61P 17/00; A61K 36/61

(57) ABSTRACT

Provided is a composition for skin whitening, including a Syzygium formosum extract as an active ingredient. The Syzygium formosum extract has excellent tyrosinase inhibitory activity, and thus can be usefully used as a cosmetic composition for skin whitening or a composition for preventing or alleviating melanin hyperpigmentation diseases. In addition, the Syzygium formosum extract has excellent antibacterial and antiatopic activities, thereby being useful as an antibacterial and antiatopic composition.

### TECHNICAL FIELD

The present disclosure relates to a cosmetic composition for skin whitening, antibacterial or antiatopic activities, including a Syzygium formosum extract as an active ingredient.

### BACKGROUND ART

Interest in skin whitening is gradually increased due to an increase in attentions for health and beauty. Preference for white skin is increased due to skin damage caused by ultraviolet rays, and clearer skin with transparent makeup is pursued. Accordingly, interest in a method for obtaining skin whitening effects is increasing.

Skin color depends on various factors such as melanin, blood vessel distribution, hemoglobin, thickness of a stratum corneum, and carotene, and from among these, melanin mainly determines the skin color. Melanin protects the skin from ultraviolet (UV) rays and physical and chemical external toxic materials, but when produced excessively, melanin causes serious beauty problems such as melasma, freckles, hyperpigmentation, etc., and also promotes skin aging, and causes skin cancer.

Melanin is produced by melanin biosynthesis in melanosomes contained in melanocytes and is regulated by melanin producing enzymes such as tyrosinase, tyrosinase related protein-1 (TRP-1) and tyrosinase related protein-2 (TRP-2). In more detail, in keratinocytes and melanocytes, α-melanocyte stimulating hormone (α-MSH) binds to a cell membrane receptor by ultraviolet rays and the amount of cyclic adenosine monophosphate (cAMP) is increased, thereby inducing the activity of protein (kinase A) and tyrosinase. Tyrosinase converts tyrosine into DOPA and dopaquinone, and the dopaquinone is converted into dopachrome by an automatic oxidase reaction and, by TRP-1 and TRP-2 (Vincent, J), melanin is generated (Vincent, J. H., et al., Int. J. Biochem., 19, 1141-1147, 1987).

Skin whitening is defined as brightening and lightening skin. The mechanism of whitening is divided into several stages, and by suppressing or blocking the process of melanin synthesis, excessive deposition of melanin on skin is suppressed and the amount of pigment in skin is reduced. Examples of such methods include inhibition of expression of tyrosinase gene, inhibition of glycosylation with respect to tyrosinase enzyme, inhibition of tyrosinase enzyme activity, promotion of tyrosinase enzyme degradation, inhibition of melanin delivery from melanocytes to keratinocytes, direct cytotoxicity with respect to melanocytes, and whitening caused by removal of keratin and acceleration of keratin formation cycle. Therefore, in order to evaluate skin whitening, it is necessary to investigate whether melanin formation is inhibited (Hearing, V). J., J Dermatol Sci., 37(1), 3-14, 2005).

In order to treat or alleviate excessive melanin pigmentation such as melasma, freckles, and pigmentation by UV exposure, etc., ascorbic acid, kojic acid, arbutin, hydroquinone, glutathione, and a substance having tyrosinase inhibitory activity are used in combination with cosmetics or medicines. However, their use is limited due to insufficient whitening effects, safety problems against skin, and formulation and stability problems when combined with cosmetics (Draelos, Z). D. Dermatol Ther., 20(5), 308-313, 2007). Therefore, in order to solve the problems of the active ingredients, research into active ingredients derived from a natural substance having proven stability is required.

Syzygium formosum is an evergreen tree that lives in Southeast Asia such as Bangladesh, India, Myanmar, Thailand, Laos, and Vietnam, and grows to a height of 10 m. In Vietnamese and Laos, Syzygium formosum is cultivated to use fruits thereof as food. In addition, in Vietnam, water extract obtained by adding water to dried leaves of Syzygium formosum is used to treat atopic skin diseases and gastrointestinal disorders.

Meanwhile, the related arts associated with a composition for skin whitening comprising Syzygium formosum extract include: the prior art [Thuong, P T et al., Natural Product Sciences, 12(1), 29-37, 2006] disclosing antioxidant activity of plant extracts including a Syzygium formosum extract; Korean Patent Publication No. 10-2013-0068307 disclosing a composition for inhibiting 15-hydroxyprostaglandin dehydrogenase (15-PGDH) including a plant extract including Syzygium formosum as an active ingredient; Korean Patent Registration No. 10-1704996 disclosing a composition for preventing or treating allergic diseases including a Syzygium formosum extract; and the prior art [Adhikari, A. et al., Int J Cosmet Sci., 30(5), 353-360, 2008] disclosing the tyrosinase inhibitory activity of Syzygium aromaticum and Syzygium cumini.

However, unlike in the present disclosure, the tyrosinase inhibitory effect and the melanin production inhibitory effect, that is, the whitening effect, of the Syzygium formosum extract, have not been described.

Also, there is no mention of antibacterial activity and antiatopic activity.

(Patent Document 0001) Korean Patent No. 10-1704996, a composition for preventing or treating allergic diseases comprising a Syzygium formosum extract, and registered on Feb. 03, 2017.

(Patent Document 0002) Korean Patent Publication No. 10-2013-0068307, a composition for inhibiting 15-hydroxyprostaglandin dehydrogenase (15-PGDH) containing a plant extract as an active ingredient, published on Jun. 26, 2013.

(Non-Patent Document 0001) Adhikari, A. et al., Screening of Nepalese crude drugs traditionally used to treat hyperpigmentation: in vitro tyrosinase inhibition, Int J Cosmet Sci., 30(5), 353-360, 2008.

(Non-Patent Document 0002) Draelos, Z. D., Skin lightening preparations and the hydroquinone controversy, Dermatol Ther., 20(5), 308-313, 2007.

(Non-Patent Document 0003) Hearing, V. J., Biogenesis of pigment granules: a sensitive way to regulate melanocyte function, J Dermatol Sci., 37(1), 3-14, 2005.

(Non-Patent Document 0004) Thuong, P. T. et al., Antioxidant Activities of Vietnamese Medicinal Plants, Natural Product Sciences, 12(1), 29-37, 2006.

(Non-Patent Document 0005) Vincent, J. H., et al., Mammalian tyrosinase-the critiacal regulatory control point in melanocyte pigmentation, Int J Biochem., 19, 1141-1147, 1987.

### DETAILED DESCRIPTION

### Technical Tasks

One or more embodiments include a skin whitening, antibacterial or antiatopic composition including a Syzygium formosum extract as an active ingredient.

One or more embodiments include a pharmaceutical composition for preventing or alleviating melanin hyperpigmentation diseases, including a Syzygium formosum extract as an active ingredient.

One or more embodiments include a pharmaceutical composition for preventing or alleviating atopic dermatitis, including a Syzygium formosum extract as an active ingredient.

### Solution

Additional aspects will be set forth in part in the description which follows and, in part, will be apparent from the description, or may be learned by practice of the presented embodiments.

According to one or more embodiments, a cosmetic composition for skin whitening, antibacterial or antiatopic activities, includes a Syzygium formosum extract as an active ingredient.

In an embodiment, in the cosmetic composition for skin whitening, antibacterial or antiatopic activities, the Syzygium formosum extract is extracted using a 50% (v/v) to 70% (v/v) aqueous ethanol solution.

In an embodiment, in the cosmetic composition for skin whitening, antibacterial or antiatopic activities, the Syzygium formosum extract is first extracted using purified water at a temperature of 60° C. to 90° C., and then secondarily extracted using a 50% (v/v) to 70% (v/v) aqueous ethanol solution.

In an embodiment, in the cosmetic composition for skin whitening, antibacterial or antiatopic activities, the Syzygium formosum extract is first extracted using purified water at room temperature and then secondarily extracted using ethanol.

In an embodiment, in the cosmetic composition for skin whitening, antibacterial or antiatopic activities, the Syzygium formosum extract may inhibit tyrosinase activity.

In an embodiment, in the cosmetic composition for skin whitening, antibacterial or antiatopic activities, the Syzygium formosum extract may inhibit the production of melanin.

In an embodiment, in the cosmetic composition for skin whitening, antibacterial or antiatopic activities, the Syzygium formosum extract may have an antibacterial activity against Staphylococcus aureus.

In an embodiment, in the cosmetic composition for skin whitening, antibacterial or antiatopic activities, the Syzygium formosum extract is an active ingredient, and may include at least one selected from Madecassic acid, Asiatic acid, Corosolic acid, Maslinic acid, and Betulinic acid.

In an embodiment, in the cosmetic composition for skin whitening, antibacterial or antiatopic activities, the active ingredient including at least one of Madecassic acid, Asiatic acid, Corosolic acid, Maslinic acid, and Betulinic acid, may be included in an amount of 10,000 ppm to 200,000 ppm based on the solid content of the Syzygium formosum extract.

The present disclosure provides a pharmaceutical composition for preventing or alleviating melanin hyperpigmentation diseases, including a Syzygium formosum extract as an active ingredient.

The present disclosure provides a pharmaceutical composition for preventing or alleviating atopic dermatitis, including a Syzygium formosum extract as an active ingredient.

### Advantageous Effects

The present disclosure relates to a composition for skin whitening including a Syzygium formosum extract as an active ingredient. The Syzygium formosum extract has excellent tyrosinase inhibitory activity and melanin production inhibitory activity, and thus can be usefully used as a cosmetic composition for skin whitening or a composition for preventing or alleviating melanin hyperpigmentation diseases.

In addition, the Syzygium formosum extract has excellent antibacterial and antiatopic activities, thereby being useful as an antibacterial and antiatopic composition.

### BRIEF DESCRIPTION OF THE DRAWINGS

FIGS. 1-4 are pictures showing the effect of improving atopic dermatitis of Syzygium formosum extract according to an embodiment of the present invention.

### BEST MODE OF EMBODIMENTS

Hereinafter, embodiments of the present disclosure will be described in detail. However, the present disclosure is not limited to the embodiments described herein and may be embodied in other forms. Rather, the embodiments enable the content disclosed herein to be thorough and complete, and are provided to a person skilled in the art in order to fully convey the concept of the present disclosure.

The cosmetic composition for skin whitening, antibacterial or antiatopic activities according to an embodiment of the present disclosure includes a Syzygium formosum extract as an active ingredient.

The Syzygium formosum extract may be obtained by extracting Syzygium formosum with water, a lower C1 to C4 alcohol, or a mixed solvent thereof, and the lower C1 to C4 alcohol may be selected from methanol, ethanol, propanol, isopropanol, butanol, and isobutanol. In an embodiment, the Syzygium formosum extract may be an extract extracted using a 50%(v/v) to 70%(v/v) aqueous ethanol solution.

In an embodiment, the extract may be obtained by hot-water extraction by using purified water at a temperature of 60° C. to 90° C., and then secondarily extracting the same at a temperature of 30° C. to 50° C. by using 50% (v/v) to 70% (v/v) aqueous ethanol solution.

In an embodiment, the extract may be obtained through a first extraction using purified water at room temperature and then a second extraction using ethanol at room temperature.

As will be described later, the extraction amount of a specific active ingredient may vary according to the extraction method, and the content ratio of the specific active ingredient may vary.

The extraction time of the extract is not particularly limited, and may be from 10 minutes to 1 day, and an extraction device may be any extraction device of the related art, an ultrasonic pulverization extractor, or a fractionation device.

In an embodiment, the Syzygium formosum extract shows a skin whitening effect by inhibiting activity of tyrosinase.

In an embodiment, the Syzygium formosum extract has an excellent skin whitening effect by inhibiting the production of melanin.

In an embodiment, the Syzygium formosum extract shows excellent antibacterial activity against Staphylococcus aureus. Staphylococcus aureus is known as a cause of atopic dermatitis, and in the following Examples, atopic dermatitis has been improved in patients with atopic dermatitis. Accordingly, it is confirmed that the antiatopic activity of the Syzygium formosum extract is excellent.

The Syzygium formosum extract, which is the active ingredient, may include, as major components, at least one of madecassic acid, Asiatic acid, Corosolic acid, Maslinic acid, and Betulinic acid, and the active ingredient including at least one of Madecassic acid, Asiatic acid, Corosolic acid, Maslinic acid, and Betulinic acid may be included in an amount of 10,000 ppm to 200,000 ppm based on the solid content of the Syzygium formosum extract to provide more excellent activity.

In the cosmetic composition, the amount of the Syzygium formosum extract may be added in an amount of 0.001 wt % to 50 wt %, or 0.001 wt % to 40 wt %, or 0.001 wt % to 30 wt %, based on the total weight of the cosmetic composition.

The cosmetic composition may be prepared in any formulation conventionally prepared in the art, for example, solutions, emulsions, suspensions, pastes, creams, lotions, gels, powders, sprays, surfactant-containing cleansing, oil, soap, liquid cleaner, bath agent, foundation, makeup base, essence, skin lotion, foam, pack, softening water, sunscreen cream, or sun oil, but is not limited thereto.

In addition, the cosmetic composition may include all other components generally used in cosmetics, in addition to the Syzygium formosum extract. For example, general auxiliary components such as emulsifiers, thickeners, emulsions, surfactants, lubricants, alcohols, water-soluble polymers, gelling agents, stabilizers, vitamins, inorganic salts, emulsifiers, and fragrances may be included. These components may be selected according to a formulation or a use purpose within an extent at which an addition amount thereof does not lead to damage of an inherent effect of a cosmetic material.

The cosmetically acceptable carrier included in the cosmetic composition of the present disclosure varies depending on the formulation of the cosmetic composition.

When the formulation of the present disclosure is a solution or emulsion, a solvent, a solubilizing agent or an emulsifying agent may be used as a carrier component, and for example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan, and the like may be used.

When the formulation of the present disclosure is a suspension, a liquid diluent such as water, ethanol or propylene glycol, a suspension agent such as ethoxylated isostearyl alcohol, polyoxyethylene sorbitol ester and polyoxyethylene sorbitan ester, microcrystalline cellulose, aluminum metahydroxide, bentonite, or tragacanth may be used as the carrier component.

When the formulation of the present disclosure is a paste, a cream or a gel, animal oil, vegetable oil, wax, paraffin, starch, tragacanth, cellulose derivatives, polyethylene glycol, silicone, bentonite, silica, talc, zinc oxide, or the like may be used as a carrier component.

When the formulation of the present disclosure is a powder or a spray, lactose, talc, silica, aluminum hydroxide, calcium silicate, polyamide powder, and the like may be used as the carrier component, and particularly, when the formulation is a spray, the formulation may further include a propellant such as chlorofluorofluorocarbon, propane/butane, or dimethyl ether.

When the formulation of the present disclosure is a surfactant-containing cleansing agent, the carrier component may include aliphatic alcohol sulfate, aliphatic alcohol ether sulfate, sulfosuccinic monoester, isethionate, imidazolium derivatives, methyltaurate, sarcosinate, fatty acid amide ether sulfate, alkylamidobetaine, aliphatic alcohol, fatty acid glycerides, fatty acid diethanolamide, vegetable oils, lanolin derivatives, or ethoxylated glycerol fatty acid esters.

The cosmetic composition containing the Syzygium formosum extract may be used on a daily basis, may be used for an indefinite period. In an embodiment, depending on the age, skin condition, or skin type of a user, and the concentration of the Syzygium formosum extract, the amount of use, the number of uses, and the period may vary.

Another aspect of the present invention provides a pharmaceutical composition for preventing or alleviating melanin hyperpigmentation diseases, including the Syzygium formosum extract as an active ingredient. The present disclosure also provides a pharmaceutical composition for preventing or alleviating atopic dermatitis, including the Syzygium formosum extract as an active ingredient.

The melanin hyperpigmentation disease may be a disease selected from freckles, chloasma, melasma, brown or black spots, gestational brown spots, senile spots, spots caused by exposure to ultraviolet rays, hyperpigmentation after inflammation caused by wounds or dermatitis, and hyperpigmentation after use of drugs.

Atopic dermatitis is a chronic recurring skin disease, often accompanied by itching, and the etiology of atopic dermatitis is not yet clear, but it is known that atopic dermatitis is caused by immunological abnormality mediated by Type 2 T-helper lymphocytes, and is also caused by environmental allergy, genetic predisposition, psychological factors, or pharmacological factors. However, the atopic dermatitis is not limited by the description provided herein.

The pharmaceutical composition according to the present disclosure may be formulated in a suitable form together with a generally used pharmaceutically acceptable carrier. The term “pharmaceutically acceptable” refers to a composition that is physiologically acceptable and does not cause an allergic reaction such as gastrointestinal disorders and dizziness or a reaction similar thereto when administered to a human.

In addition, the pharmaceutical composition may be formulated and used in oral forms such as powders, granules, tablets, capsules, suspensions, emulsions, syrups, and aerosols, in skin external applications, suppositories, and sterile injectable solutions, according to conventional methods. The carrier, excipient, and diluent that may be included in the pharmaceutical composition may include lactose, dextrose, sucrose, sorbitol, mannitol, xylitol, erythritol, maltitol, starch, gum arabic, alginate, gelatin, calcium phosphate, calcium silicate, cellulose, methyl cellulose, microcrystalline cellulose, polyvinyl pyrrolidone, water, methyl paraoxybenzoate, propyl paraoxybenzoate, talc, magnesium stearate, and mineral oil, but are not limited thereto. The formulation may be performed using a diluent or excipient such as a filler, a stabilizer, a binder, a disintegrant, a surfactant, and the like, which are generally used. The solid formulations for oral administration include tablets, pills, powders, granules, capsules, and the like, and the solid formulations are prepared by mixing the extract of the present disclosure with at least one excipient such as starch, calcium carbonate, sucrose or lactose, gelatin, and the like. In addition to the simple excipients, lubricants such as magnesium stearate and talc may also be used. Liquid formulations for oral administration include suspensions, solutions, emulsions, syrups, and the like, and various other excipients such as wetting agents, sweeteners, fragrances, and preservatives may be included in addition to water and liquid paraffin which are commonly used simple diluents. Formulations for parenteral administration include sterile aqueous solutions, non-aqueous solvents, suspensions, emulsions, lyophilized formulations, or suppositories. Examples of the non-aqueous solvent and the suspension are propylene glycol, polyethylene glycol, vegetable oil such as olive oil, and injectable ester such as ethylolate. As a base for suppositories, witepsol, macrogol, tween 61, cacao butter, laurin paper, glycerogelatin, and the like may be used.

The pharmaceutical composition including the Syzygium formosum as an active ingredient may be administered to mammals such as rats, livestock, and humans via various routes. All modes of administration may be contemplated, e.g., by oral, rectal or intravenous, muscle, subcutaneous, intrauterine dura or intracerebral injection. The dosage may vary depending on the age, sex, weight of the subject to be treated, the specific disease or pathology to be treated, the severity of the disease or pathology, the time of administration, the route of administration, the absorption, distribution and excretion rate of the drug, the type of other drugs to be used, and the judgment of the prescriber. The dosage determined based on these factors are within the level of those skilled in the art, and the dosage may be, in general, in the range of 0.001 mg/kg/day to 2000 mg/kg/day. The dosage may be from 0.01 mg/kg/day to 500 mg/kg/day. The administration may be performed once or several times a day. The dosage does not limit the scope of the present disclosure in any way.

### EXAMPLE 1 AND EXAMPLE 2

### Preparation of Syzygium formosum Extract

To 20 g of Syzygium formosum dry powder, 200 ml of 50% (v/v) aqueous ethanol solution or 70% (v/v) aqueous ethanol solution was added, followed by extraction at 40° C. for 2 hours. The extract obtained from the above process was filtered and concentrated to obtain a Syzygium formosum 50% aqueous ethanol solution extract of Example 1 and a Syzygium formosum 70% aqueous ethanol solution extract of Example 2.

### COMPARATIVE EXAMPLE 1

### Preparation of Syzygium formosum 10% Aqueous Ethanol Solution Extract

A Syzygium formosum 10% aqueous ethanol solution extract was obtained in the same manner as in Example 1, except that a 10% aqueous ethanol solution was used instead of 50% aqueous ethanol solution.

### COMPARATIVE EXAMPLE 2

### Preparation of Syzygium formosum 20% Aqueous Ethanol Solution Extract

A Syzygium formosum 20% aqueous ethanol solution extract was obtained in the same manner as in Example 1, except that a 20% aqueous ethanol solution was used instead of 50% aqueous ethanol solution.

### COMPARATIVE EXAMPLE 3

### Preparation of Syzygium formosum 40% Aqueous Ethanol Solution Extract

A Syzygium formosum 40% aqueous ethanol solution extract was obtained in the same manner as in Example 1, except that a 40% aqueous ethanol solution was used instead of 50% aqueous ethanol solution.

### COMPARATIVE EXAMPLE 4

### Preparation of Syzygium formosum 80% Aqueous Ethanol Solution Extract

A Syzygium formosum 80% aqueous ethanol solution extract was obtained in the same manner as in Example 1, except that a 80% aqueous ethanol solution was used instead of 50% aqueous ethanol solution.

### COMPARATIVE EXAMPLE 5

### Preparation of Syzygium formosum Ethanol Extract

A Syzygium formosum 100% ethanol extract was obtained in the same manner as in Example 1, except that a 100% ethanol was used instead of 50% aqueous ethanol solution.

### COMPARATIVE EXAMPLE 6

### Preparation of Syzygium formosum Methanol Extract

A Syzygium formosum 100% methanol extract was obtained in the same manner as in Example 1, except that a 100% methanol was used instead of 50% aqueous ethanol solution.

### COMPARATIVE EXAMPLE 7

### Preparation of Syzygium formosum Hot Water Extract

A Syzygium formosum 100% hot-water extract was obtained in the same manner as in Example 1, except that the extraction was performed using water at a temperature of 80° C. for 1 hour using water, instead of 50% aqueous ethanol solution.

### EXPERIMENTAL EXAMPLE 1

### Confirmation of Tyrosinase Inhibitory Activity using L-Tyrosine

The inhibitory activity of tyrosinase, which is an enzyme that plays an important role in the melanin synthesis process, was confirmed using the Syzygium formosum extract.

500 μl of 1 mM L-tyrosine, 900 μl of 0.1 M phosphate buffer (pH 6.8), and 100 μl of 0.5 mg/ml Syzygium formosum extract dissolved in 10% DMSO (Example 1, Example 2, and Comparative Examples 1 to 7) were mixed, and then 100 μl of tyrosinase (53.7 U/ml) was added thereto and reacted at a temperature of 25° C. for 15 minutes. At this time, DMSO was used as a control group, and 0.5 mg/ml arbutin was used as a positive control group.

The absorbance of the reaction solution was measured at 475 nm for 15 minutes at time intervals of 30 seconds, and then, the tyrosinase inhibitory activity (%) was calculated using Equation 1.

$$Inhibition ( \% ) = 100 - [ \frac{{slope}_{sample}}{{slope}_{control}} ] \times 100 \qquad Equation 1$$

TABLE 1

| Condition | | Inhibitory activity (%) |
|---|---|---|
| Example 1 | 50% aqueous ethanol | 75.0 |
| | solution extract | |
| Example 2 | 70% aqueous ethanol | 70.8 |
| | solution extract | |
| Comparative Example 1 | 10% aqueous ethanol | 25.3 |
| | solution extract | |
| Comparative Example 2 | 20% aqueous ethanol | 31.2 |
| | solution extract | |
| Comparative Example 3 | 40% aqueous ethanol | 40.0 |
| | solution extract | |
| Comparative Example 4 | 80% aqueous ethanol | 54.5 |
| | solution extract | |
| Comparative Example 5 | 100% ethanol extract | 40.7 |
| Comparative Example 6 | 100% methanol extract | 30.4 |
| Comparative Example 7 | Hot water extract | 20.0 |
| Positive control group | Arbutin | 49.1 |
| Control group | DMSO | 0 |

As shown in Table 1, it was confirmed that the Syzygium formosum 50% aqueous ethanol solution and the Syzygium formosum 70% aqueous ethanol solution respectively prepared according to Examples 1 and 2 of the present disclosure had excellent tyrosinase inhibitory activities compared to arbutin, which is the positive control group. In particular, Examples of the present disclosure showed remarkably excellent tyrosinase inhibitory activity compared to the Syzygium formosum extracts of Comparative Examples 1 to 7 of which solvent conditions were different from those of Examples.

In addition, although not shown in Table 1, from among plants belonging to the same genus as but different species from Syzygium formosum used in the present disclosure, Syzygium aromaticum and Syzygium cumini showed 20 times lower the tyrosinase inhibitory activity than the Syzygium formosum extract. Accordingly, it was confirmed that even in the same genus of plants, the difference in tyrosinase inhibitory activity value thereof was remarkable.

Therefore, from the above results, it can be seen that the Syzygium formosum extracts of Examples 1 and 2 according to the present disclosure are usefully used as a composition showing the skin whitening effect by suppressing melanin formation in the skin.

### EXPERIMENTAL EXAMPLE 2

### Confirmation of Tyrosinase Inhibitory Activity Using L-DOPA

Tyrosinase inhibitory activity was confirmed using L-DOPA.

(1) Preparation of Reagents
Tyrosinase solution: Tyrosinase was dissolved in 0.1M PBS (Phosphate buffered saline) buffer to prepare a solution having a concentration of 110 U/ml.
Substrate: L-DOPA was dissolved in 0.1M PBS buffer to be in the concentration of 5 mM.
Sample Usage: Diluted with distilled water to a corresponding concentration and used.
Negative control: Distilled water was added instead of the sample.

(2) Test Methods

Syzygium formosum extract (Example 1, Example 2, and Comparative Examples 1 to 7), 30 μl of negative control (control), 30 μl of positive control (positive control), 70 μl of 0.1M PBS buffer, and 30 μl of substrate were added. Then, 20 μl of tyrosinase was added thereto, and the reaction was performed at a temperature of 25° C. for 5 minutes. After completion of the reaction, absorbance was measured at 475 nm. The tyrosinase inhibitory activity (%) was calculated using Equation 1 by comparing the absorbance of the test group with that of the control group. Results thereof are shown in Table 2.

TABLE 2

| Condition | | Inhibitory activity (%) |
|---|---|---|
| Example 1 | 50% aqueous ethanol | 32.3 |
| | solution extract | |
| Example 2 | 70% aqueous ethanol | 46.2 |
| | solution extract | |
| Comparative Example 1 | 10% aqueous ethanol | 8.7 |
| | solution extract | |
| Comparative Example 2 | 20% aqueous ethanol | 15.9 |
| | solution extract | |
| Comparative Example 3 | 40% aqueous ethanol | 24.8 |
| | solution extract | |
| Comparative Example 4 | 80% aqueous ethanol | 28.5 |
| | solution extract | |
| Comparative Example 5 | 100% ethanol extract | 27.7 |
| Comparative Example 6 | 100% methanol extract | 24.4 |
| Comparative Example 7 | Hot water extract | 8.5 |
| Positive control group | Arbutin | 18.6 |
| Control group | Distilled water | 0 |

As shown in Table 2, it was confirmed that the Syzygium formosum 50% aqueous ethanol solution and the Syzygium formosum 70% aqueous ethanol solution respectively prepared according to Examples 1 and 2 of the present disclosure had excellent tyrosinase inhibitory activities compared to arbutin, which is the positive control group.

### EXPERIMENTAL EXAMPLE 3

### Confirmation of Melanin Production Inhibitory Activities

(1) Cell Culture

The cell line used in the test was B16F10 cell line (Korean Cell Line Bank, Korea), which is a malignant melanoma cell in mice. The cryopreserved cell line was cultured in a culture dish, and after cell line stabilization was confirmed while repeating the subculture twice to three times in the cycle of 2 to 3 days after the first cell culture, cells were cultured in a 48-well plate by the number of cells of 1×10⁵/well. Then, the test was prepared by incubating for 24 hours.

(2) Sample Preparation

A test was prepared by dissolving the Syzygium formosum extracts (Example 1, Example 2, and Comparative Examples 1 to 7) in dimethyl modified eagle medium (DMEM, 10-013-CVR, Corning, USA) at a concentration of 0.1 mg/ml.

(3) Test Method

300 μl of the medium containing each of the sample, the negative control group, and the positive control group was added to a 48-well plate on which cell had been cultured in advance, and cell were cultured for 72 hours at 37° C. under 5% CO₂ while the medium was exchanged every day. Thereafter, the medium was removed by washing each well with PBS, and then the cells were treated with 200 μl of 1N NaOH. Thereafter, absorbance was measured at 400 nm using a SpectraMax M2 (Molecular devices, USA). The degree of inhibition on the production of melanin was calculated by comparing the absorbance of the test group with that of the control group treated with the negative control group by using Equation 2. Results thereof are shown in Table 2.

$$Inhibition ( \% ) = 100 - [ \frac{{slope}_{sample}}{{slope}_{control}} ] \times 100 \qquad Equation 2$$

TABLE 3

| Condition | | Inhibitory activity (%) |
|---|---|---|
| Example 1 | 50% aqueous ethanol | 22.8 |
| | solution extract | |
| Example 2 | 70% aqueous ethanol | 28.6 |
| | solution extract | |
| Comparative Example 1 | 10% aqueous ethanol | 5.6 |
| | solution extract | |
| Comparative Example 2 | 20% aqueous ethanol | 10.5 |
| | solution extract | |
| Comparative Example 3 | 40% aqueous ethanol | 18.6 |
| | solution extract | |
| Comparative Example 4 | 80% aqueous ethanol | 20.6 |
| | solution extract | |
| Comparative Example 5 | 100% ethanol extract | 19.2 |
| Comparative Example 6 | 100% methanol extract | 18.9 |
| Comparative Example 7 | Hot water extract | 4.7 |
| Positive control group | Arbutin | 21.1 |
| Control group | DMEM | 0 |

Therefore, from the above results, it can be seen that the Syzygium formosum extracts of Examples 1 and 2 according to the present disclosure are usefully used as a composition having the skin whitening effect by suppressing melanin formation in the skin.

### EXPERIMENTAL EXAMPLE 4

### Confirmation of Antibacterial Activity

The antibacterial activity was tested using the Syzygium formosum extract of Example 2. As the target strain, Staphylococcus aureus (S. aureus), which is a skin opportunistic infectious bacteria and an atopic causative bacteria, was selected to confirm antibacterial activity and antiatopic activity.

(1) S. Preparation of aureus Strain

First, Staphylococcus aureus (hereinafter referred to as S. aureus) strain was cultured in LB media at a temperature of 37° C. at 200 rpm. aureus), and then, diluted with the bacterial number of 8×10⁶ cfu/ml using physiological saline to prepare the strain.

(2) Preparation of Syzygium formosum Extract

The Syzygium formosum extract of Example 2 was dissolved in DMSO, and diluted, and 30 μl thereof was added to LB to finally prepare a sample of Syzygium formosum extract at concentrations of 0.1, 0.2, 0.25, 0.4, 0.5, 0.6, 0.75, 1, and 1.5 mg/ml.

(3) Antibacterial Activity Test

The Syzygium formosum extract sample was inoculated with 30 μl of S. aureus strain to make the number of bacteria be 8×10⁶ cfu/ml, and then cultured for 12 hours at 37° C. and at 120 rpm, and the absorbance thereof at 595 nm was measured to test the antibacterial activity. 5% DMSO was used as a control group, and the results are shown in Table 4.

TABLE 4

| | Control | | | | | | |
|---|---|---|---|---|---|---|---|
| | (final | | | | | | |
| | 5% | S. formosume (mg/ml) | | | | | |
| | DMSO) | 0.1 | 0.25 | 0.5 | 0.75 | 1 | 1.5 |
| 1 | 0.656 | 0.596 | 0.489 | 0.439 | 0.265 | 0.208 | −0.065 |
| 2 | 0.696 | 0.606 | 0.426 | 0.268 | 0.421 | 0.304 | 0.272 |
| 3 | 0.821 | 0.627 | 0.555 | 0.345 | 0.453 | 0.148 | −0.203 |
| 4 | 0.657 | 0.593 | 0.499 | 0.435 | 0.312 | 0.190 | 0.331 |
| 5 | 0.723 | 0.648 | 0.529 | 0.378 | 0.295 | 0.237 | 0.029 |
| aver- | 0.711 ± | 0.614 ± | 0.500 ± | 0.373 ± | 0.349 ± | 0.217 ± | 0.073 ± |
| age | 0.068 | 0.023 | 0.048 | 0.071 | 0.083 | 0.058 | 0.226 |

As shown in Table 4, it can be seen that the Cythium formosum extract according to an embodiment of the present disclosure has excellent antibacterial activity and antiatopic activity.

### EXAMPLE 3 TO EXAMPLE 6

### Confirmation of Components of Syzygium formosum Extract according to Extraction Conditions

### EXAMPLE 3

200 ml of 70% (v/v) aqueous ethanol solution was added to 20 g of Syzygium formosum dry powder, followed by extraction at room temperature for 2 hours. The extract obtained from the above-described process was filtered and concentrated to obtain a Syzygium formosum extract.

### EXAMPLE 4-1

200 ml of purified water was added to 20 g of Syzygium formosum dry powder, followed by extraction for 1 hour at a temperature of 80° C. Then, the result was filtered, and concentrated to obtain a Syzygium formosum extract.

### EXAMPLE 4-2

200 ml of 50% (v/v) aqueous ethanol solution was added to the Syzygium formosum filtered in Example 4-1, and the extraction process was performed at a temperature of 40° C. for 2 hours. The extract solution obtained from the above-described process was filtered and concentrated to obtain a Syzygium formosum extract.

### EXAMPLE 4-3

200 ml of 70% (v/v) aqueous ethanol solution was added to the Syzygium formosum filtered in Example 4-2, and the extraction process was performed at a temperature of 40° C. for 2 hours. The extract solution obtained from the above-described process was filtered and concentrated to obtain a Syzygium formosum extract.

### EXAMPLE 5-1

200 ml of 70% (v/v) aqueous ethanol solution was added to 20 g of Syzygium formosum dry powder, followed by extraction at room temperature for 1 hour. The extract solution obtained from the above-described process was filtered and concentrated to obtain a Syzygium formosum extract.

### EXAMPLE 5-2

200 ml of 70% (v/v) aqueous ethanol solution was added to the Syzygium formosum filtered in Example 5-1, and the extraction process was performed at room temperature for 1 hour. The extract solution obtained from the above-described process was filtered and concentrated to obtain a Syzygium formosum extract.

### EXAMPLE 5-3

200 ml of 70% (v/v) aqueous ethanol solution was added to the Syzygium formosum filtered in Example 5-2, and the extraction process was performed at room temperature for 1 hour. The extract solution obtained from the above-described process was filtered and concentrated to obtain a Syzygium formosum extract.

### EXAMPLE 6-1

200 ml of purified water was added to 20 g of dry Syzygium formosum dry powder, followed by extraction at room temperature for 30 minutes. Then, the result was filtered, and concentrated to obtain a Syzygium formosum extract.

### EXAMPLE 6-2

200 ml of about 100% ethanol was added to the Syzygium formosum filtered in Example 4-1, and the extraction process was performed at room temperature for 2 hour. The extract solution obtained from the above-described process was filtered and concentrated to obtain a Syzygium formosum extract.

### EXPERIMENTAL EXAMPLE 5

### Confirmation of Components of Syzygium formosum Extract using HRMS Analysis

(1) HRMS Analysis Conditions

HRMS analysis was performed under the conditions of Tables 5 and 6 below.

TABLE 5
HRMS condition

| | |
|---|---|
| Column | YMC - Triart C18 (5 um 250 × 4.6 mm) |
| Solvent | (A) 0.1% Formic acid in water |
| | (B) 0.1% Formic acid in CAN |
| Flow rate | 0.5 ml/min |
| Injection volume | 10 ul |

TABLE 6

| Time (min) | A(%) | B(%) |
|---|---|---|
| 0 | 75 | 25 |
| 25 | 60 | 40 |
| 65 | 10 | 90 |
| 66 | 0 | 100 |
| 70 | 0 | 100 |
| 75 | 75 | 25 |
| 80 | 75 | 25 |

* * solvent gradient over time

(2) Quantification Results of Major Components of the Active Ingredient

Major components of the active ingredient of the Syzygium formosum extract of Example 3 were identified, and measurements of the weights thereof are shown in Table 7 based on the solid content of the Syzygium formosum extract.

TABLE 7

| Main components of active | Amount |
|---|---|
| ingredient | (ppm) |
| Madecassic acid | 16000 |
| Asiatic acid | 15000 |
| Corosolic acid | 48000 |
| Maslinic acid | 36000 |
| Betulinic acid | 12000 |

(3) Analysis Result of active Ingredient Extraction Efficiency according to Extraction Conditions

The extraction efficiency of active ingredients according to the extraction conditions of Examples 3 to 6 was analyzed by HRMS.

TABLE 8

| | Madecassic | Asiatic | Corosolic | Maslinic | Betulinic |
|---|---|---|---|---|---|
| | acid | acid | acid | acid | acid |
| Example 3 | 100 | 100 | 100 | 100 | 100 |
| Example 4-1 | 2 | 20 | 0 | 0 | 0 |
| Example 4-2 | 16 | 71 | 55 | 53 | 23 |
| Example 4-3 | 14 | 78 | 31 | 29 | 22 |
| Example 5-1 | 21 | 60 | 40 | 38 | 35 |
| Example 5-2 | 32 | 0 | 0 | 0 | 31 |
| Examples | 26 | 0 | 0 | 0 | 11 |
| 5-3 | | | | | |
| Example 6-1 | 2 | 15 | 0 | 0 | 0 |
| Example 6-2 | 20 | 52 | 30 | 28 | 35 |

### PREPARATION EXAMPLE 1

### Preparation of Cosmetic Composition

### FORMULATION EXAMPLE 1-1

### Preparation of Softening Skin Lotion

By using the Syzygium formosum 50% aqueous ethanol solution extract of Example 1 of the present disclosure, softening skin lotion was prepared by a conventional method according to the composition shown in Table 9 below.

TABLE 9

| Source name | Content (wt %) |
|---|---|
| Example 1 | 4.0 |
| Butylene glycol | 3.5 |
| Glycerin | 2.5 |
| Polyoxyethylene cured castor oil | 0.1 |
| Ethanol | 2.5 |
| Betaine | 1.0 |
| Citric acid | 0.01 |
| Sodium citrate | 0.03 |
| Antiseptic | Appropriate amount |
| spice | Appropriate amount |
| Purified water | to 100 |

### FORMULATION EXAMPLE 1-2

### Manufacture of Nutritional Essence

By using the Syzygium formosum 50% aqueous ethanol solution extract of Example 1 of the present disclosure, a nutritional essence was prepared by a conventional method according to the composition shown in Table 10 below.

TABLE 10

| Source name | Content (wt %) |
|---|---|
| Example 1 | 7.0 |
| Cetostearyl alcohol | 1.0 |
| Self-emulsifying monostearate | 1.0 |
| Beeswax | 0.5 |
| Squalane | 5.0 |
| Isocetyloctanoate | 3.0 |
| Dimethylsiloxane | 0.3 |
| Sorbitan monostearate | 0.5 |
| Polyethylene glycol monostearate | 8.0 |
| Glycerin | 4.0 |
| Propylene glycol | 0.2 |
| Carboxypolymer | 0.22 |
| Triethanolamine | 0.25 |
| Antiseptic | Appropriate amount |
| fragrances | Appropriate amount |
| Coloring agent | Appropriate amount |
| Purified water | to 100 |

### FORMULATION EXAMPLE 1-3

### Preparation of Cream

By using the Syzygium formosum 50% aqueous ethanol solution extract of Example 1 of the present disclosure, a cream was prepared by a conventional method according to the composition shown in Table 11 below.

TABLE 11

| Source name | Content (wt %) |
|---|---|
| Example 1 | 7.0 |
| Cetostearyl alcohol | 3.0 |
| Self-emulsifying monostearate | 1.5 |
| Lipophilic monostearic acid | 1.5 |
| Beeswax | 0.5 |
| Floating paraffin | 8.0 |
| Squalane | 7.0 |
| Isocetyloctanoate | 4.0 |
| Purified jojoba oil | 4.0 |
| Dimethylsiloxane | 0.3 |
| Sorbitan monostearate | 1.0 |
| Polyethylene glycol monostearate | 1.2 |
| Glycerin | 6.0 |
| Propylene glycol | 4.0 |
| Betaine | 4.0 |
| Xanthan gum | 0.06 |
| Triethanolamine | 0.10 |
| Antiseptic | 0.25 |
| fragrances | Appropriate amount |
| Coloring agent | Appropriate amount |
| Purified water | to 100 |

### EXPERIMENTAL EXAMPLE 6

### Antiatopic Activity Test

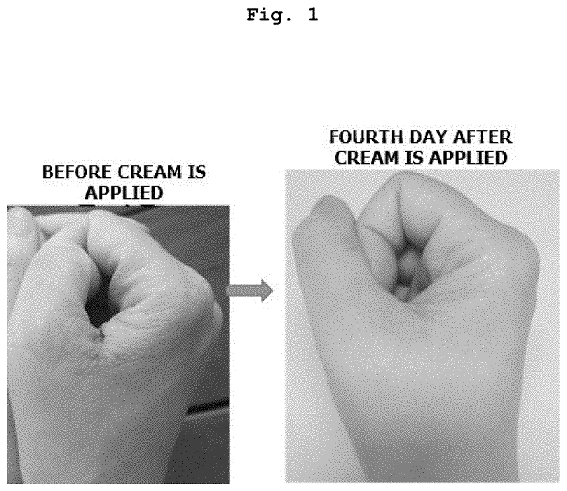

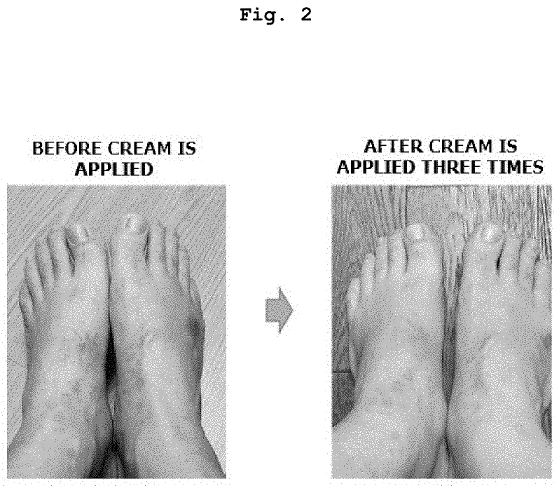

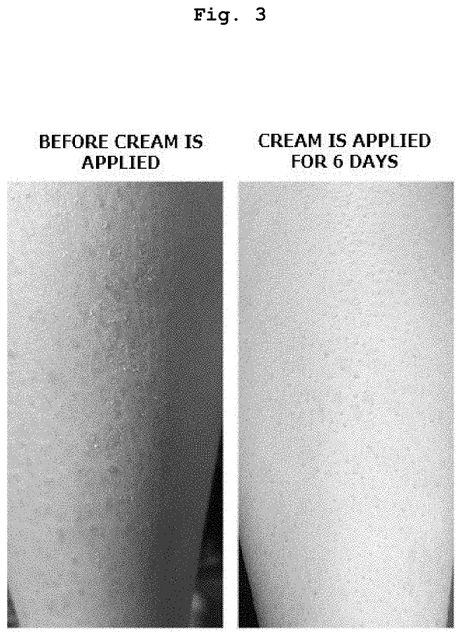

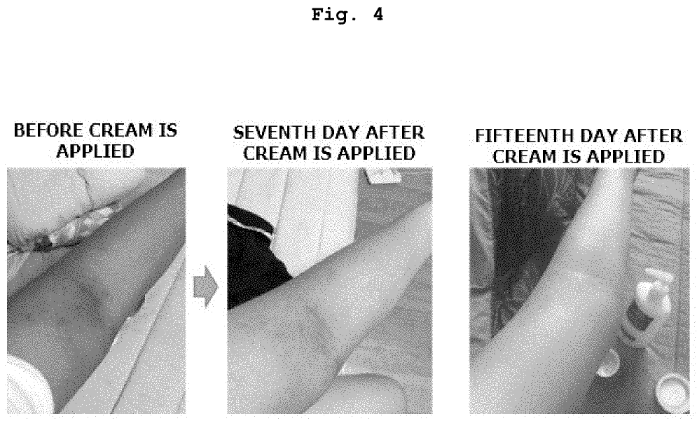

For patients with acute exacerbated atopic dermatitis, the cream of Preparation Examples 1 to 3 was applied in an appropriate amount on the affected parts of atopic patients three times a day, and then, the alleviation effect of atopic dermatitis was confirmed. Results thereof are shown in FIGS. 1 to 4. From the results, it can be seen that atopic symptoms have been significantly improved.

It should be understood that embodiments described herein should be considered in a descriptive sense only and not for purposes of limitation. Descriptions of features or aspects within each embodiment should typically be considered as available for other similar features or aspects in other embodiments.

What is claimed is:

1. A cosmetic composition for skin whitening, antibacterial or antiatopic activities, the cosmetic composition comprising a Syzygium formosum extract as an active ingredient.

2. The cosmetic composition of claim 1, wherein the Syzygium formosum extract is extracted using a 50% (v/v) to 70% (v/v) aqueous ethanol solution.

3. The cosmetic composition of claim 1, wherein the Syzygium formosum extract is first extracted using purified water at a temperature of 60° C. to 90° C., and then secondarily extracted using a 50% (v/v) to 70% (v/v) aqueous ethanol solution.

4. The cosmetic composition of claim 1, wherein the Syzygium formosum extract is first extracted using purified water at room temperature and then secondarily extracted using ethanol.

5. The cosmetic composition of claim 1, wherein the Syzygium formosum extract inhibits the activity of tyrosinase.

6. The cosmetic composition of claim 1, wherein the Syzygium formosum extract inhibits the production of melanin.

7. The cosmetic composition of claim 1, wherein the Syzygium formosum extract has an antibacterial activity against Staphylococcus aureus.

8. The cosmetic composition of claim 1, wherein the Syzygium formosum extract used as the active ingredient, comprises at least one selected from Madecassic acid, Asiatic acid, Corosolic acid, Maslinic acid, and Betulinic acid.

9. The cosmetic composition of claim 8, wherein the active ingredient including at least one of Madecassic acid, Asiatic acid, Corosolic acid, Maslinic acid, and Betulinic acid, is included in an amount of 10,000 ppm to 200,000 ppm based on the solid content of the Syzygium formosum extract.

10. A pharmaceutical composition for preventing or alleviating melanin hyperpigmentation diseases, the pharmaceutical composition comprising a Syzygium formosum extract as an active ingredient.

11. A pharmaceutical composition for preventing or alleviating atopic dermatitis, the pharmaceutical composition comprising a Syzygium formosum extract as an active ingredient.
